(19) United States
(12) Patent Application Publication
(10) Pub. No.: US 2011/0015604 A1
(43) Pub. Date: Jan. 20, 2011

(54) URINE ABSORBENT POUCH FOR MALE INCONTINENCE

(75) Inventor: Lucas Bäck, Billdal (SE)

Correspondence Address: BUCHANAN, INGERSOLL & ROONEY PC, POST OFFICE BOX 1404, ALEXANDRIA, VA, 22313-1404, US

(73) Assignee: SCA HYGIENE PRODUCTS AB, GOTEBORG (SE)

(21) Appl. No.: 12/667,160

(22) PCT Filed: Jul. 9, 2007
(86) PCT No.: PCT/SE07/50515

§ 371 Date: Dec. 29, 2009

(51) Int. Cl. A61F 13/45

(52) U.S. Cl. 604/385.03

(57) ABSTRACT

A urine absorbent pouch for male incontinence includes an absorbent core enclosed between two casing sheets. The casing sheets are extended beyond the lateral sides of the absorbent core and beyond the opposite longitudinal ends thereof and are attached to each other in the parts extending beyond the absorbent core. The unit of casings sheets and enclosed absorbent core are folded around a lateral fold line so that two parts of the unit are folded in over each other. The two pairs of attached casing sheets extending beyond the respective lateral side of the folded unit are sealed to each other by a seam extending from the fold line in a longitudinal direction and ending a distance from the ends of the folded unit. The two parts of the unit include a through-going hole, attaching means, and an elastic member provided along the peripheral edge of the hole.

### TECHNICAL FIELD

The present invention relates to a urine absorbent pouch for male incontinence comprising an absorbent core enclosed between two casing sheets, the casing sheets being extended beyond the lateral sides of the absorbent core and beyond the opposite longitudinal ends thereof and being attached to each other in the parts extending beyond the absorbent core, the unit of casings sheets and enclosed absorbent core being folded around a lateral fold line so that two parts of said unit are folded in over each other, the two pairs of attached casing sheets extending beyond the respective lateral side of said folded unit being sealed to each other by a seam extending from said fold line in a longitudinal direction and ending a distance from the ends of said folded unit, wherein a through-going hole is present in one of said two parts of said unit and one of said parts includes means for attaching the longitudinal edges and/or lateral edges of said parts to each other, an elastic member being provided along the peripheral edge of said hole.

### BACKGROUND OF THE INVENTION

Urine absorbent pouches or bags for male incontinence in which the penis of the user is inserted into the pouch through a hole in the wall thereof are known, see EP 0 861 643 A2 and U.S. Pat. No. 6,569,135 B1. Due to the effectiveness of modern absorbents, such as so called SAP (superabsorbent particles), such pouches can be made small and discreet and they also are comfortable to wear compared to incontinence protectors shaped like large diapers or pouches in which both scrotum and penis are enclosed. In order to hold the pouch in place it is suggested to arrange elastic elements around the periphery of the hole.

However, the dimensions of the hole can not be chosen so that all penis sizes will have a close fit in the hole. In order to create a sufficient tension around an inserted penis, the hole must be expanded, even for a small penis. This means that the tension in the material surrounding the hole easily can be too large for a comfortable wearing of the pouch and the tension can also create skin irritation.

The objective of the present invention is to provide a pouch of the kind mentioned in the introduction which is comfortable to wear for all penis sizes, is securely held in place on the wearer and reduces the risk for leakage to a minimum.

### SUMMARY OF THE INVENTION

These objectives are accomplished by a urine absorbent pouch for male incontinence comprising an absorbent core enclosed between two casing sheets, the casing sheets being extended beyond the lateral sides of the absorbent core and beyond the opposite longitudinal ends thereof and being attached to each other in the parts extending beyond the absorbent core, the unit of casings sheets and enclosed absorbent core being folded around a lateral fold line so that two parts of said unit are folded in over each other, the two pairs of attached casing sheets extending beyond the respective lateral side of said folded unit being sealed to each other by a seam extending from said fold line in a longitudinal direction and ending a distance from the ends of said folded unit, wherein a through-going hole is present in one of said two parts of said unit and one of said parts includes means for attaching the longitudinal edges and/or lateral edges of said parts to each other, an elastic member being provided along the peripheral edge of said hole, characterised in that each pair of attached casing sheets extended in the longitudinal direction from the respective longitudinal end of the absorbent core forms a flap, said hole being provided in one of said flaps and the elastic member is a patch of elastic material covering said hole in said flap, said patch having a opening therein with a perimeter smaller than the perimeter of the hole in said flap, every point on the perimeter of the opening being distanced at least 10 mm from the edge of said hole. In such a pouch, the elastic material will form a sleeve around a penis inserted into the pouch through the opening in the patch of elastic material. The elastic force needed to securely hold the penis will be distributed over the whole area of the sleeve formed by the insertion of the penis. The material in said piece can thereby be chosen so that the force per area unit acting on the penis after elongation of the elastic material caused by the insertion of the penis though said opening is small which make said the pouch very comfortable to wear. Moreover, the size of the hole in the flap can be made larger than largest penis size since the penis is held by said sleeve of elastic material so it is easy to dimension such a pouch to fit all penis sizes. Since the penis is held by a sleeve, a large sealing surface is obtained reducing the risk for fluid leaking out of the opening

In a preferred embodiment wherein the force needed to elongate the patch of elastic material 100% is less than 6 N/25 mm. The patch of elastic material consists preferably of an elastic film or of elastic foam.

Said casing sheets are advantageously rectangular which facilitates manufacturing of such pouches with a minimum of waste.

Said seams are preferably extended to a longitudinal end of the absorbent core and the absorbent core is preferably folded into two halves and the flaps have the same extension from the respective longitudinal end of the absorbent core to their respective longitudinal end. By such a design the part of the pouch into which the penis is inserted is securely sealed and the parts extending outside the penis are thin and flexible and thereby very discrete.

The patch of elastic material is attached to one of said flaps and the patch of elastic material is preferably located on the inside of the flap, i.e. the side distal from the wearer during use of the pouch.

### BRIEF DESCRIPTION OF THE DRAWINGS

The invention will now be described with reference to the enclosed Figures, of which;

FIG. 1 is a schematic plan view of a preferred embodiment of a pouch according to the invention, and

FIG. 2 schematically discloses a production line for manufacturing the pouch according to FIG. 1.

### DESCRIPTION OF EMBODIMENTS

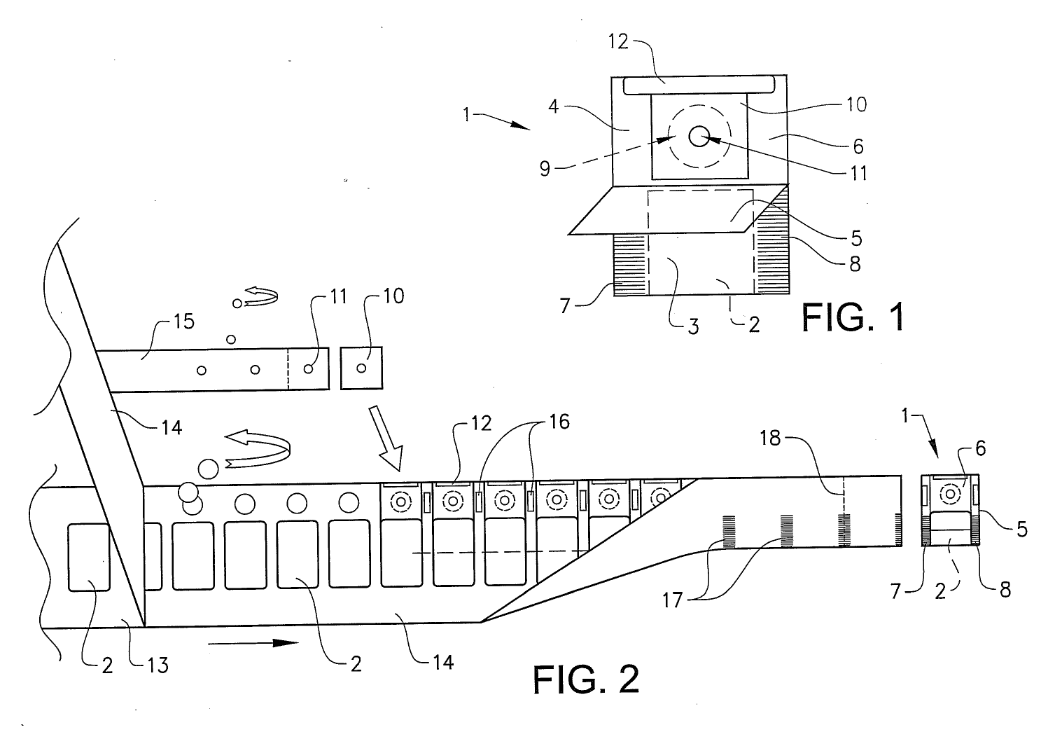

A urine absorbing pouch 1 for male incontinence according to a preferred embodiment of the invention is schematically disclosed in FIG. 1.

The pouch consists of an absorbent core 2 enclosed between two casing sheets 3,4. The casing sheets 3,4 extend beyond the core 2 in all directions and are extended from the longitudinal end edges of the absorbent core 2 to form flaps 5,6. The casing sheets 3,4 are attached to each other in the portions extending beyond the absorbent core 2. The unit consisting of the casing sheets 3,4 and the absorbent core 2 enclosed therebetween is in the embodiment of FIG. 1 double-folded so that the longitudinal end edges of the absorbent core 2 are aligned to each other and also the longitudinal edges of the flaps 5,6. The opposite lateral edges of said double-folded unit are sealed to each other by seams 7,8 that extend longitudinally from the fold up to the longitudinal end edges of the absorbent core. Thereby a sealed bag is formed, the walls of which containing absorbent material.

In the centre of flap 6 a circular hole 9 is present and a rectangular patch 10 of elastic material is affixed to the inside of flap 6. Said patch 10 has a circular opening 11 therein which is concentric to hole 9 and much smaller.

On the inside of flap 6 is, at least along the longitudinal end edge thereof, arranged a strip 12 of adhesive which before use is covered by a release layer.

In use of the pouch 1, penis is inserted through opening 11 in the patch 10 of elastic material. The opening 11 should be dimensioned so that it will be expanded to a certain extent also by a small penis so that a sleeve of elastic material always will surround a part of the penis after insertion into the pouch. The elastic material should be a low tension material so that the pressure exerted on the penis by the expanded sleeve of elastic material after insertion will be low. The elasticity of the material should be such that a force less than 6 N/25 mm is needed to elongate the material 100%, said force being measured in accordance with ASTM D882. By such a material the pressure exerted on the penis will not be felt uncomfortable or skin irritating even for large penis sizes. After insertion of the penis into the pouch 1, the release paper on the adhesive string 12 is removed and the longitudinal edges of the flaps 5,6 are brought together and fastened to each other. This contributes to ensure that the penis is maintained in the sealed bag having walls containing absorbent material. Since the flaps contain no absorbent material they are very flexible and easily conformable to the body of the wearer. This contributes to making the pouch 1 very discreet when worn.

The hole 9 in the flap 6 can be made so large, for example have a diameter of 45 mm, so that a penis independent of its size can pass theretrough since a inserted penis will be maintained in the pouch by the elastic force of the sleeve of elastic material surrounding a portion of the penis. A pouch according to the present invention can thus easily be dimensioned to fit all penis sizes as long as the opening 11 in the patch 10 is so small that it is expanded at least 500% or more when a penis is inserted theretrough. The opening 11 should have a diameter that is at least 10 mm smaller than the diameter of the hole 9 in flap 6.

The absorbent material in the core 2 is preferably a mix of cellolosic fibres, fluff, and SAP, for example a mix of 50% fluff and 50% SAP. The SAP chosen should preferably be a SAP with a large storing capacity, such as HySorb B7160 from BASF, Ludwigshafen, Germany. The density of the core should be greater than 120 g/dm³ in order to create a good fluid distribution with the core. The sealed bag of the pouch having such a mix of absorbent material in its wall shall have an adequate size so that the volume thereof can accommodate a momentarily emitted gush of urine. An appropriate volume is 0.15 l. In order to satisfy this requirement the area of the absorbent core should be greater than 60 cm². However, this area should preferably not be larger than 144 cm² in order to ensure that the pouch will be discrete when worn.

The inner casing sheet 4 should be liquid-permeable and all materials used for liquid-permeable top sheets of disposable hygienic absorbent articles, such as incontinence protectors, diapers, sanitary napkins and the like are possible to use as inner sheet of the pouch 1. The inner casing sheet can be hydrophilic or hydrophobic and consist of a single layer or multiple layers. One example of a suitable material is a hydrophilic spunbond nonwoven from BBA Fiberweb, Norrkoping, Sweden having a basis weight of 15 g/m².

The outer casing sheet 3 can be liquid-impermeable and all materials used for liquid-impermeable backing sheets of disposable hygienic absorbent articles, such as incontinence protectors, diapers, sanitary napkins and the like are possible to use as outer casing sheet of the pouch 1. Preferably, the casing sheet 3 is breathable having a vapour permeability of about 2500 g/m²/24 h or more. Like the inner casing sheet 3 can consist of one or more layers and preferably the outermost layer is a layer having a textile-like appearance. One example of a suitable material for the outer casing sheet is a laminate of a breathable film and a nonwoven available from Nuova Pansac, Milan, Italy under the trade named Mira Air33B11 having a vapour permeability of 3000 g/m²/24 h.

The pouch 1 can also be used as an insert in a larger incontinence protector for adults and in such a case the outer casing sheet should be liquid-permeable.

The patch 10 of elastic material is preferably made of a thin elastic film or an elastic foam. However, also other materials having the required weak elasticity can be used. One example of a suitable material for the patch 10 is an elastic film from Nordenias, Gronau, Germany sold under the trade name KC6325 having a basis weight of 49 g/m² and which need a force of 3 N/25 mm for elongation 100%.

The casing sheets 3,4 are preferably attached to each other in the parts extending outside the absorbent core 2 by a glue pattern of for example a hot melt adhesive but other adhesives or other ways of attaching the sheets to each other can be used, such as ultrasonic bonding or application of heat and pressure. Also the seams 7,8 can consist of a glue seam or a seam produced by ultra sonic or heat bonding.

The strip of adhesive 12 on an edge or on edges is preferably a pressure sensitive adhesive (PSA) of the type used on sanitary napkins for attaching such articles to underwear. Before use strips 12 are protected by a conventional release tape.

In FIG. 2, a production line for the manufacture of pouches according to the above described embodiment is schematically shown.

In said line, a row of absorbent cores 2 are laid upon a web 13 of material suitable for the backing sheet of the final product. Said web is placed on an appropriate conveyer moving in the direction of the arrow in FIG. 2. Thereafter a web 14 suitable for the inner casing sheet of the final product is laid onto the unit of web 13 and row of absorbent cores 2 and the webs are attached to each other by a pattern of glue points in areas outside the cores 1 in the row of cores. In order to ensure the attachment of the webs to each other, the unit of the two webs 13,14 with cores 2 enclosed therebetween passes a pair of rollers (not shown in FIG. 2).

After the attaching of the casing sheet webs 13,14 to each other, a hole 9 is cut through the webs 13,14 in an area which in FIG. 2 is located above each absorbent core 2. Parallel to the webs 13, 14, a web 15 of elastic material is fed in the same direction as the webs 13,14. At a location upstream of the cutting device cutting the holes 9 in webs 13,14, individual patches 10 is cut out of the web 15 and are attached to these webs covering the hole 9. Before the cutting of web 15, a row of openings 11 are cut in the web 15. The device for application of the patches 10 preferably includes a spray gun or the like for the application of a glue pattern on each patch before the application threof onto the web 14. Said glue applicator can of course be arranged to apply the glue pattern on the web 15 before the cutting of individual patches 10.

After application of the patches 10 of elastic material to the web 14, adhesive strings 12 are applied along the upper edge of each patch and optionally is adhesive strings 16 applied along the lateral side edges of each patch. Said strings of adhesive are then covered by release tapes.

Thereafter, the unit consisting of the webs 13,14, the row of absorbent cores 2 enclosed therebetween, the row of elastic patches attached to web 14 and the adhesive strings 12,16 covered by release tapes is double folded so that the opposite edges of the webs 13,14 running in the fed direction of said webs are aligned to each other. The dimensions and placing of the cores 2 between the webs 13,14 is such that the upper and lower edges of the cores, as seen in FIG. 2, also will be aligned to each other after said double folding. Thereafter a row of seams 17 extended along the lateral sides of the folded cores 2 are made and individual pouches 1 are finally cut out of the webs by cutting along cutting lines 18 dividing each seam 17 in two.

The individual pouches 1 are then packaged folded, preferably by folding the flaps 5,6 in over the double folded absorbent core 2 and put into a package.

The devices in the production line in FIG. 2 are all components known from the manufacture of incontinence protectors, diapers, sanitary napkins and the like and form no part of the invention and need therefore not be described in detail for an understanding of the present invention.

The production line of FIG. 2 can of course be modified in several ways, for example can the openings 11 in the patches 10 be made after the patches are attached to webs 13,14. The web 15 of elastic material need not run parallel to the webs 13,14 but can run perpendicular thereto.

As is evident from the description above of the production line in FIG. 2, pouches 1 can be manufactured with a minimum of waste, the only waste being the material removed when cutting the holes 9 and openings 11, respectively.

The pouch according to the invention is thus very discreet, comfortable to wear, easy to manufacture with a minimum of waste, allows a compact packaging thereof and reduces the risk for leakage to a minimum.

The described embodiment can of course be modified without leaving the scope of invention. The dimensions can be different, for example can the longitudinal end of the absorbent core being opposite to the flap containing the elastic patch be extended beyond the opposite longitudinal edge of the absorbent core. Even if it is preferred that the hole in one of the parts of the folded unit of casing sheets and enclosed absorbent core is circular other shapes, as rectangular or oval, are possible. Also the opening in the patch of elastic material need not be circular even if this is preferred. Even if it is preferred that the pouch is rectangular in the folded condition, other shapes are possible. The patch of elastic material need not be rectangular, even if this is preferred in order to save waste. Other fastening devices than adhesive strings for attaching the flaps to each other can be used, for example hooks and loops fasteners. The absorbent core can consist of several layers of absorbent material instead of one layer of mixed SAP and fluff. The invention should therefore only be limited by the content of the enclosed claims.

1. A urine absorbent pouch for male incontinence comprising an absorbent core enclosed between two casing sheets, the casing sheets being extended beyond the lateral sides of the absorbent core and beyond the opposite longitudinal ends thereof and being attached to each other in the parts extending beyond the absorbent core to form a unit of casing sheets and the enclosed absorbent core, the unit of casings sheets and the enclosed absorbent core being folded around a lateral fold line so that two parts of said unit are folded in over each other, the two pairs of attached casing sheets extending beyond the respective lateral sides of said folded unit being sealed to each other by a seam extending from said fold line in a longitudinal direction and ending a distance from the ends of said folded unit, wherein a through-going hole is present in one of said two parts of said unit and one of said parts includes an attachment device adapted for attaching the longitudinal edges and/or lateral edges of said parts to each other, an elastic member being provided along the peripheral edge of said hole, wherein each pair of attached casing sheets extended in the longitudinal direction from the respective longitudinal end of the absorbent core forms a flap, said hole is provided in one of said flaps and the elastic member is a patch of elastic material covering said hole in said flap, said patch having an opening therein with a perimeter smaller than the perimeter of the hole in said flap, and every point on the perimeter of the opening being distanced at least 10 mm from the edge of said hole.

2. The pouch according to claim 1, wherein the force needed to elongate the patch of elastic material 100% is less than 6 N/25 mm.

3. The pouch according to claim 2, wherein the patch of elastic material consists of an elastic film.

4. The pouch according to claim 2, wherein the patch of elastic material consists of elastic foam.

5. The pouch according to claim 1, wherein said casing sheets are rectangular.

6. The pouch according to claim 1, wherein said seams are extended to a longitudinal end of the absorbent core.

7. The pouch according to claim 5, wherein the absorbent core is folded into two halves and the flaps have the same extension from the respective longitudinal end of the absorbent core to their respective longitudinal end.

8. The pouch according to claim 7, wherein the patch of elastic material is attached to one of said flaps.

9. The pouch according to claim 8, wherein the patch of elastic material is located on the inside of the flap, which is the side distal from the wearer during use of the pouch.

10. The pouch according to claim 6, wherein the absorbent core is folded into two halves and the flaps have the same extension from the respective longitudinal end of the absorbent core to their respective longitudinal end.

11. The pouch according to claim 10, wherein the patch of elastic material is attached to one of said flaps.

12. The pouch according to claim 11, wherein the patch of elastic material is located on the inside of the flap, which is the side distal from the wearer during use of the pouch.
